(19) United States
(12) Patent Application Publication
(10) Pub. No.: US 2025/0090136 A1
(43) Pub. Date: Mar. 20, 2025

(54) METHODS, SYSTEMS, AND COMPUTER READABLE MEDIA FOR USING TRAINED MACHINE LEARNING MODEL INCLUDING AN ATTENTION MODULE TO ESTIMATE GESTATIONAL AGE FROM ULTRASOUND IMAGE DATA

(71) Applicant: The University of North Carolina at Chapel Hill, Chapel Hill, NC (US)

(72) Inventors: Jeffrey Samuel Allen Stringer, Chapel Hill, NC (US); Juan Carlos Prieto Bernal, Carrboro, NC (US); Teeranan Pokaprakarn, Raleigh, NC (US)

(21) Appl. No.: 18/720,482

(22) PCT Filed: Dec. 23, 2022
(86) PCT No.: PCT/US2022/053924

§ 371 Date: Jun. 14, 2024

Related U.S. Application Data
(60) Provisional application No. 63/293,439, filed on Dec. 23, 2021.

(51) Int. Cl. A61B 8/08; A61B 8/00; G06N 3/0464

(52) U.S. Cl. CPC A61B 8/0866; A61B 8/5223; G06N 3/0464

(57) ABSTRACT

A method for estimating gestational age of a human fetus using a trained machine learning model with an attention function includes receiving, at a feature extraction module of a trained machine learning model, fetal ultrasound image data for at least one image of a human fetus, and producing, by propagating the ultrasound image data through the feature extraction module, at least one feature vector from the ultrasound image data. The method further includes providing the at least one feature vector as input to an attention module of the trained machine learning model and producing, by propagating the feature vectors through the attention module, a weighted sum vector that aggregates and weights the feature vectors. The method further includes providing the weighted sum vector as input to a gestational age prediction module of the trained machine learning model, which generates, from the weighted sum vector, an estimate of the gestational age of the human fetus. The method further includes outputting the estimate of gestational age to a user.

### PRIORITY CLAIM

This application claims the priority benefit of U.S. Provisional Patent Application Ser. No. 63/293,439 filed Dec. 23, 2021, the disclosure of which is incorporated herein by reference in its entirety.

### TECHNICAL FIELD

The subject matter described herein relates to estimating gestational age of human subjects. More particularly, the subject matter described herein relates to methods, systems and computer readable media for estimating gestational age of a human subject using a trained machine learning model including an attention module to estimate gestational age from ultrasound image data.

### BACKGROUND

Gestational age is established as early as feasible in pregnancy and then used to determine the timing of subsequent care. Providers use gestational age to interpret abnormalities of fetal growth, make referral decisions, intervene for fetal benefit, and time delivery. By convention, gestational age is expressed as the time elapsed since the start of the last menstrual period (LMP). Although easily solicited, self-reported LMP has long been recognized as problematic. Some women may be uncertain of the LMP date. Some (perhaps most) will have a menstrual cycle that varies from the “normal” 28-day length with ovulation on day 14. It is therefore best practice to confirm gestational age dating with an ultrasound exam in early pregnancy. This is achieved by fetal biometry, the measuring of standard fetal structures and applying established formulas.

Although ubiquitous in industrialized regions, obstetric ultrasound is infrequently used in low- and middle-income countries. Reasons for this disparity include the expense of traditional ultrasound machines, their requirement of reliable electrical power, the need for trained obstetrics trained sonographers to obtain images, and the need for expert interpretation. However, two recent developments offer solutions to these obstacles. The first is the availability of low-cost, battery-powered ultrasound devices. There are now more than a dozen manufacturers of low cost probes that can be used with a smart phone or tablet. The second innovation is recent advances in the field of computer vision. Deep learning algorithms are increasingly capable of interpreting radiologic images and these models can be deployed on mobile devices.

In light of the need for non-expert methods for estimating gestational age and the availability of machine learning algorithms, there exists a need for a machine learning algorithm that can receive as input ultrasound image data, including ultrasound image data collected by a non-expert, and that can accurately estimate gestational age of a human subject.

### SUMMARY

A method for estimating gestational age of a human fetus using a trained machine learning model with an attention function includes receiving, at a feature extraction module of a trained machine learning model, fetal ultrasound image data for at least one image of a human fetus, and producing, by propagating the ultrasound image data through the feature extraction module, at least one feature vector from the ultrasound image data. The method further includes providing at least one feature vector as input to an attention module of the trained machine learning model and producing, by propagating the feature vector(s) through the attention module, a weighted sum vector that aggregates and weights the feature vector(s). The method further includes providing the weighted sum vector as input to a gestational age prediction module of the trained machine learning model, which generates, from the weighted sum vector, an estimate of the gestational age of the human fetus. The method further includes outputting the estimate of gestational age to a user. A scoring mechanism from the attention module determines whether the gestational age estimate is sufficiently reliable to be output to a user.

The subject matter described herein can be implemented in software in combination with hardware and/or firmware. For example, the subject matter described herein can be implemented in software executed by a processor. In one exemplary implementation, the subject matter described herein can be implemented using a non-transitory computer readable medium having stored thereon computer executable instructions that when executed by the processor of a computer control the computer to perform steps. Exemplary computer readable media suitable for implementing the subject matter described herein include non-transitory computer-readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF THE DRAWINGS

Exemplary implementations and test results of the subject matter described herein will now be explained with reference to the accompanying drawings of which:

FIG. 1 is a block diagram illustrating exemplary training and test datasets used to train and test a machine learning model to estimate gestational age;

FIG. 2 illustrates graphs of gestational age predictions made by a trained machine learning model compared to predictions made by a trained sonographer;

FIG. 3 illustrates graphs of gestational age predictions made by the trained machine learning model as compared to predictions made by un-trained users for a novice test set;

FIG. 4 illustrates one example of an architecture for a trained machine learning model for predicting gestational age from blind sweep ultrasound data;

FIG. 5 illustrates graphs of the gestational age distributions of the training and test data sets described in FIG. 1;

FIG. 6 is a flow diagram illustrating the steps performed in a participant visit and ultrasound data collection in the FAMLI study;

FIG. 7 is a block diagram illustrating a trained machine learning module that can be used to estimate gestational age from ultrasound sweep data;

FIG. 8 is a flow chart illustrating an exemplary process for estimating gestational age from ultrasound sweep data;

FIG. 9 is a block diagram illustrating an alternate machine learning model for estimating gestational age from ultrasound sweep data;

FIG. 10 shows the steps to create 2 embeddings of the same image under different transformations;

FIG. 11 is a diagram illustrating a 128-dimension unit hypersphere; and

FIG. 12 is a diagram illustrating a confusion matrix for the classification task.

### DETAILED DESCRIPTION

This document describes a study in which ultrasound image data is used to train a machine or deep learning algorithm to estimate gestational age of human subjects. The Methods session describes the study. Section 1 of the appendix describes the machine learning model and the training of the model. Section 2 of the appendix describes an exemplary machine learning model. Section 3 of the appendix contains supplemental tables. Section 4 of the appendix describes the use of a trained machine learning model and two different model architectures to estimate gestational age.

### Methods

The Fetal Age Machine Learning Initiative (FAMLI) is an ongoing project that is developing technologies to expand obstetric ultrasound access to resource-limited settings. Prospective data collection commenced in September 2018 at two sites in Chapel Hill, North Carolina, USA and in 2 Jan. 2019 at two sites in Lusaka, Zambia. The project enrolls women who are at least 18 years old, have a confirmed intrauterine pregnancy, and provide written informed consent. The study protocol is approved by the relevant ethical authorities at the University of North Carolina and the University of Zambia.

### Sonography

Each site employs certified sonographers for ultrasound procedures. Participants are recruited during prenatal care and complete a single study visit with no required follow-up; however, we do allow repeat study visits no more frequently than bi-weekly. Evaluation is conducted with a commercial ultrasound machine (multiple makes and models; Table 4). We perform fetal biometry by crown rump length (if <14 weeks) or biparietal diameter, head circumference, abdominal circumference, and femur length (if ≥14 weeks). Each fetal structure is measured twice, and the average taken.

During the same examination we also collect a series of blind sweep cineloops. These are free-hand sweeps, approximately 10 seconds in length, across the gravid abdomen in multiple directions and probe configurations. Cranio-caudal sweeps start at the pubis and end at the level of uterine fundus with the probe indicator facing toward the maternal right either perpendicular (90 degrees) or angled (15 and 45 degrees) to the line of probe movement. Lateral sweeps are performed with the probe indicator facing superiorly, starting just above the pubis and sweeping from the left to the right lateral-uterine borders and moving cephalad to the uterine fundus. Complete sets of blind sweeps are collected by the study sonographer on both the commercial ultrasound machine and a low-cost, battery powered device (Butterfly iQ; Guilford, CT, USA). In June 2020, we began collecting a third series of sweeps at the Zambia sites. These “novice blind sweeps” are obtained by a nurse midwife with no training in sonography and include three sweeps in the cranio-caudal axis and three in the lateral axis with the low-cost probe. Prior to obtaining the sweeps, the novice measures the participant's symphysial-fundal height and sets the depth parameter on the ultrasound device as follows: fundus not palpable=11 cm depth; fundus palpable but <25 cm=13 cm depth; fundus≥25 cm=15 cm depth.

Except for a small number of participants who conceived by in vitro fertilization, ground truth gestational age is established by the first ultrasound received. At the North Carolina sites, women present early in pregnancy and gestational age is set according to the American College of Obstetricians and Gynecologists practice guidelines, which incorporate fetal biometry from the first scan and the reported LMP. At the Zambia sites, women present later in pregnancy and the LMP is less reliable. We thus assign gestational age based solely upon the results of the first scan, an approach that antedates the FAMLI protocol.

### Training, Tuning, and Testing Datasets

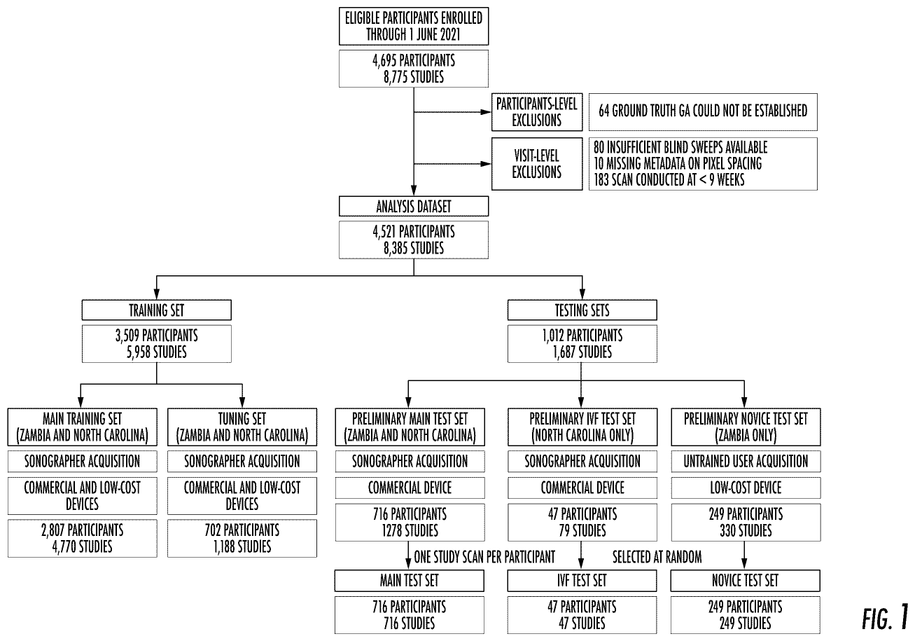

Participants with viable single pregnancies enrolled between September 2018 and June 2021 were included (FIG. 1). We applied participant-level exclusions to women whose available medical records did not allow a ground truth gestational age to be established. We applied visit-level exclusions to study scans that (a) did not contain at least two blind sweep cineloops, (b) had missing pixel spacing metadata, or (c) were conducted before 9 weeks of gestation (because they were too infrequent to allow model training). After applying exclusions, we apportioned the remaining data into five non-overlapping groups of participants to develop the deep learning model (training and tuning sets) and evaluate its performance (three test sets). FIG. 1 illustrates the creation of training and testing datasets.

After applying participant and visit-level exclusions, we created 2 training sets to develop and tune the deep learning model and 3 test sets to assess its performance. To be eligible for inclusion in a test set a participant must have been dated by a prior scan or in vitro fertilization (IVF). The IVF test set comprises all participants who conceived by IVF. The novice test set comprises all participants in whom at least one study visit included sweeps collection by a novice user on a low-cost device. The main test set was selected at random from among all remaining eligible participants Some participants apportioned to the test sets had contributed more than one study scan; in such cases we selected a single study scan at random. The training sets comprise all participants who remain after creation of the test sets and were split randomly, by participant, in a 4:1 ratio, into a main training set and a tuning set.

The three test sets were created first. The IVF test set comprises women who conceived by in vitro fertilization (and thus whose gestational age was known with certainty); all were enrolled in North Carolina. The novice test set contains participants who contributed at least one study scan from the novice blind sweep protocol; all were enrolled in Zambia. Our primary assessments are made on an independent main test set, which was created as a simple random sample of 30% of eligible women who remained after creation of the other test sets. It includes participants from both Zambia and North Carolina. After establishing the participant members of each test set, we ensured that each woman contributed only a single study scan to her respective test set through random selection (FIG. 1). Sensitivity analyses that include all participant study scans are presented in the Appendix.

To be included in a test set, a pregnancy had to be dated by either a prior ultrasound or in vitro fertilization; this establishes the ground truth against which both the deep learning model and biometry are measured. In Zambia, a single ultrasound provided by the FAMLI protocol may have been the only scan received. In North Carolina, a single ultrasound provided by the FAMLI protocol may have been conducted on the same day as the participant's clinical dating ultrasound. In such cases without a prior ground truth benchmark, comparison of the model's estimate to that of biometry is not possible. These women were thus only included in the datasets used for training. After creation of the three test sets, all remaining participants were randomly allocated in a 4:1 ratio into a main training set (80%) and a tuning set (20%).

### Technical Methods of the Deep Learning Model

Our novel, end-to-end deep learning model takes blind sweep cineloops as input and provides a gestational age estimate as output. Details of the model architecture and constituent parts, including pre-processing steps, training procedure and parameters, and inference procedure are provided in the Appendix.

TABLE 1
Characteristics of participants in the combined training and tuning sets and in the three test sets

| | Training & | | | | | | | |
|---|---|---|---|---|---|---|---|---|
| | Tuning | | Main Test | | IVF Test | | Novice Test | |
| | Sets | | Set | | Set | | Set | |
| Characteristic | N = 3509 | | N = 716 | | N = 47 | | N = 249 | |
| Age, median | 28 | (24, 33) | 29 | (25, 33) | 37 | (33, 39) | 27 | (23, 30) |
| years (IQR) | | | | | | | | |
| Body mass | 24.7 | (21.9, 28.5) | 24.8 | (22.1, 29.3) | 24.6 | (22.7, 28.1) | 24.2 | (21.8, 27.2) |
| index (kg/m2)a | | | | | | | | |
| <18 · 5 | 111 | (3.1%) | 18 | (2.5%) | 1 | (2.1%) | 7 | (2.8%) |
| 18 · 5−30 | 2552 | (72.7%) | 492 | (68.7%) | 34 | (72.3%) | 199 | (79.9%) |
| >30 | 617 | (17.6%) | 146 | (20.4%) | 7 | (14.9%) | 22 | (8.9%) |
| Parity, median | 2 | (2, 4) | 2 | (1, 4) | 2 | (1, 4) | 2 | (2, 4) |
| (IQR) | | | | | | | | |
| GA at | 23.0 | (13.7, 29.9) | 15.1 | (8.8, 24.1) | 9.8 | (8.0, 12.3) | 23.7 | (19.8, 27.4) |
| presentation, | | | | | | | | |
| median weeks | | | | | | | | |
| (IQR) | | | | | | | | |
| No. studies | 1 | (1, 13) | 2 | (1, 11) | 1 | (1, 6) | 2 | (2, 6) |
| contributed, | | | | | | | | |
| median | | | | | | | | |
| (range) | | | | | | | | |
| Chronic | 195 | (5.6%) | 35 | (4.9%) | 0 | (0%) | 11 | (4.4%) |
| hypertension, | | | | | | | | |
| n (%) | | | | | | | | |
| Diabetes, n | 60 | (1.7%) | 23 | (3.2%) | 6 | (12.8%) | 3 | (1.2%) |
| (%)** | | | | | | | | |
| HIV infection, | 721 | (20.5%) | 140 | (19.5%) | 0 | (0%) | 38 | (15.3%) |
| n (%) | | | | | | | | |
| Syphilis in | 81 | (2.3%) | 23 | (3.2%) | 0 | (0%) | 7 | (2.8%) |
| pregnancy, n | | | | | | | | |
| (%) | | | | | | | | |
| Asthma | 194 | (5.5%) | 59 | (8.2%) | 8 | (17.0%) | 3 | (1.2%) |
| Tuberculosis | 80 | (2.3%) | 14 | (2.0%) | 0 | (0%) | 2 | (0.8%) |
| Alcohol use | 416 | (11.9%) | 146 | (20.4%) | 13 | (27.7%) | 17 | (6.8%) |
| during | | | | | | | | |
| pregnancy | | | | | | | | |
| Tobacco use | 74 | (2.1%) | 37 | (5.2%) | 0 | (0%) | 0 | (0%) |
| during | | | | | | | | |
| pregnancy | | | | | | | | |
| Pregnancy | 2 | (0.1%) | 0 | (0%) | 47 | (100%) | 0 | (0%) |
| conceived by | | | | | | | | |
| in vitro | | | | | | | | |
| fertilization | | | | | | | | |

aMissing height prevents BMI calculation of 5 in IVF test set, 21 in novice test set, 60 in main test set, 230 in combined training sets
b Includes both pregestational and “prevalent gestational” (i.e., a participant who would go on to develop gestational diabetes after her last study scan is not counted as diabetic here)

### Statistical Assessment of Diagnostic Accuracy

Predictive performance of both the model and the biometry is assessed by comparing each approach's estimate to the previously established ground truth gestational age. The absolute difference between these quantities is the absolute error of the prediction. We report the mean absolute error (MAE) with its standard error (SE), along with the root mean squared error (RMSE) of each approach. We use a paired t-test to assess the mean of the pairwise difference between the model absolute error and the biometry absolute error (|Model Error|−|Biometry Error|). Our null hypothesis was that the mean of this pairwise difference is zero; a negative mean of the pairwise difference whose 95% confidence interval does not include zero would indicate the model to be superior. We compare the model MAE to that of biometry in the overall test datasets and in subsets by geography (Zambia vs North Carolina) and trimester (defined as ≤97 days, 98-195 days, ≥196 days). We also plot the empirical cumulative distribution function (CDF) for the absolute error produced by the model and the biometry. From this empirical CDF, we compare the proportion of study scans in which the absolute error is <7 days and <14 days for the model vs. biometry, using McNemar's test. Wald-type 95% confidence intervals for the difference in proportions are also computed. Finally, for the novice test set only, we present the diagnostic accuracy of LMP reported at the first patient visit, since this is the relevant comparator for implementation of this technology in low-resource settings.

### Results

From September 2018 through June 2021, 4,695 participants contributed 8,775 ultrasound studies at the four research sites (FIG. 1). After applying participant- and visit-level exclusions, we created the three test sets as follows: 712 participants (from both North Carolina and Zambia) formed the main test set; 47 participants (all from North Carolina) formed the IVF test set; 249 participants (all from Zambia) formed the novice test set. As outlined above, participants were allowed to contribute only a single study scan (chosen at random) to their respective test set. The 3,509 participants who remained after creation of the test sets were randomly apportioned into the main training and tuning sets in a 4:1 ratio. Collectively, these women contributed 5,958 study scans containing 109,806 blind sweeps containing 21,264,762 individual image frames for model training and tuning. Baseline characteristics of women included in the combined training sets and the three test sets are presented in Table 1.

TABLE 2
Gestational age estimation of deep learning model compared
to sonographer in the main test set and IVF test set

| | Main Test Set (n = 716)^{a} | | | IVF Test Set (n = 47)^{b} | | |
|---|---|---|---|---|---|---|
| | | | Difference | | | Difference |
| | Model | Biometry | (95% CI) | Model | Biometry | (95% CI) |
| Mean | 3.9 | 4.7 | −0.8 | 2.8 | 3.6 | −0.8 |
| Absolute | (0.12) | (0.15) | (−1.1, −0.5) | (0.28) | (0.53) | (−1.7, 0.2) |
| Error (SE), | | | | | | |
| DAYS | | | | | | |
| Root Mean | 5.1 | 6.1 | −1.1 | 3.4 | 5.1 | −1.7 |
| Square | (0.18) | (0.19) | (−1.5, −0.6) | (0.31) | (0.67) | (−2.8, −0.5) |
| Error, days | | | | | | |
| 1^{st} | 2.1 | 2.0 | 0.2 | 2.3 | 2.2 | |
| trimester^{c} | (0.19) | (0.23) | (−0.4, 0.7) | (0.34) | (0.38) | |
| MAE (SE), | | | | | | |
| days | | | | | | |
| 2^{nd} | 3.1 | 3.3 | −0.2 | 2.7 | 3.4 | |
| trimester^{c} | (0.16) | (0.17) | (−0.5, 0.1) | (0.45) | (0.84) | |
| MAE (SE), | | | | | | |
| days | | | | | | |
| 3^{rd} | 4.7 | 6.0 | −1.3 | 3.7 | 5.7 | |
| trimester^{c} | (0.18) | (0.22) | (−1.8, −0.8) | (0.69) | (1.38) | |
| MAE (SE), | | | | | | |
| days | | | | | | |
| Absolute | 86.0 | 77.0 | 9.1 | 95.7 | 83.0 | |
| Error <7 | (1.3) | (1.6) | (5.7, 12.5) | (2.9) | (5.5) | |
| days (SE), | | | | | | |
| % | | | | | | |
| Absolute | 98.9 | 96.9 | 2.0 | 100.0 | 100.0 | |
| Error <14 | (0.4) | (0.6) | (0.5, 3.4) | | | |
| days (SE), | | | | | | |
| % | | | | | | |
| North | 3.4 | 4.0 | −0.6 | | | |
| Carolina | (0.15) | (0.20) | (−0.9, −0.2) | | | |
| MAE (SE), | | | | | | |
| days | | | | | | |
| Zambia | 4.4 | 5.4 | −1.0 | | | |
| MAE (SE), | (0.19) | (0.22) | (−1.5, −0.5) | | | |
| days | | | | | | |

^{a}The main test set comprises a 30% random sample of participants who are dated by a prior ultrasound and who are not included in the IVF or novice test sets; participants enrolled in either North Carolina or Zambia; blind sweeps and fetal biometry were collected by a sonographer on a commercial ultrasound machine.
^{b}The IVF test set comprises all studies conducted in women who conceived by in vitro fertilization; all participants were enrolled in North Carolina; blind sweeps and fetal biometry were collected by a sonographer on a commercial ultrasound machine.
^{c}Trimesters defined as ≤97 days, 98-195 days, ≥196 days; MAE = mean absolute error

### Model Versus Biometry in the Main Test Set and IVF Test Set

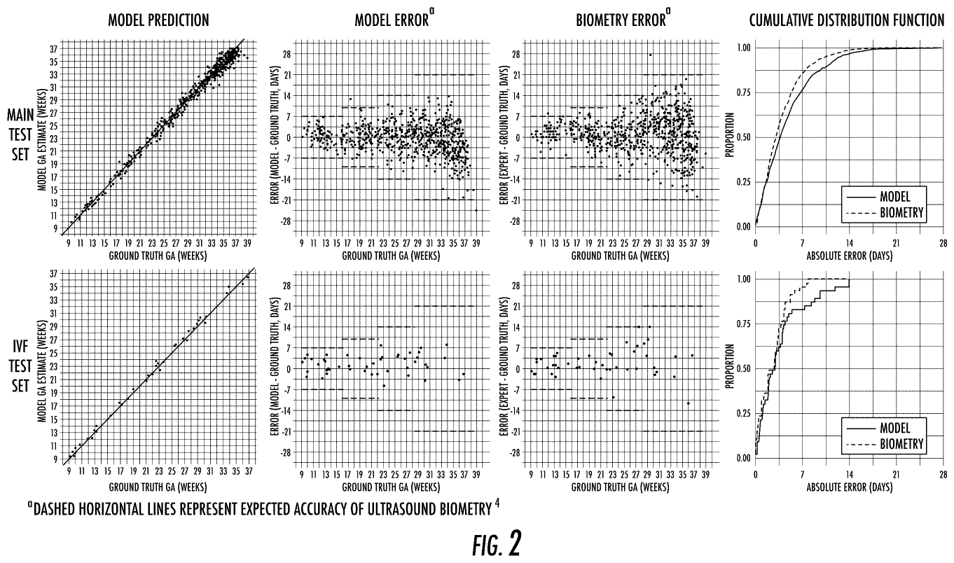

Table 2 and FIG. 2 illustrate gestational age predictions made by the machine learning model as compared to predictions made by a trained sonographer for the main test set and the IVF test set. In the main test set, the deep learning model outperformed biometry: overall model MAE 3.9 days (SE 0.12) versus biometry MAE 4.7 days (SE 0.15); difference −0.8 days (95% CI: −1.1, −0.5); p<0.001). The observed difference manifested primarily in the third trimester, where the mean of the pairwise difference in absolute error was −1.3 days (95% CI—1.8, −0.8; p<0.001). Based on the empirical cumulative distribution function (CDF), the proportion of study scans that were correctly classified within 7 days was higher for the model than for biometry (86.0% vs 77.0%; difference 9.1%; 95% CI: 5.7%, 12.5%; p<0.001). The model similarly outperformed biometry using a 14 day classification window (98.9% vs 96.9%; difference 2.0%; 95% CI: 0.5%, 3.4%; p=0.01).

Among the 47 study scans in the IVF test set, the model MAE was 2.8 days (SE 0.28) compared to MAE of 3.6 days (SE 0.53) for biometry (difference=−0.8 days; 95% CI: −1.7, 0.2; p=0.10). As was observed in the main test set, the difference was most pronounced in the third trimester where the estimated mean of the pairwise difference in absolute error was-2.0 days. Based on the empirical CDF, the proportion of study scans that were correctly classified within 7 days was higher for the model than for biometry (95.7% vs 83.0%).

Owing to the small sample size in our IVF test set, we did not perform statistical tests on the difference by trimester or the difference in proportion. Both model and biometry correctly categorized 100% of cases within 14 days (Table 2).

### Model Versus Biometry and LMP in the Novice Test Set—

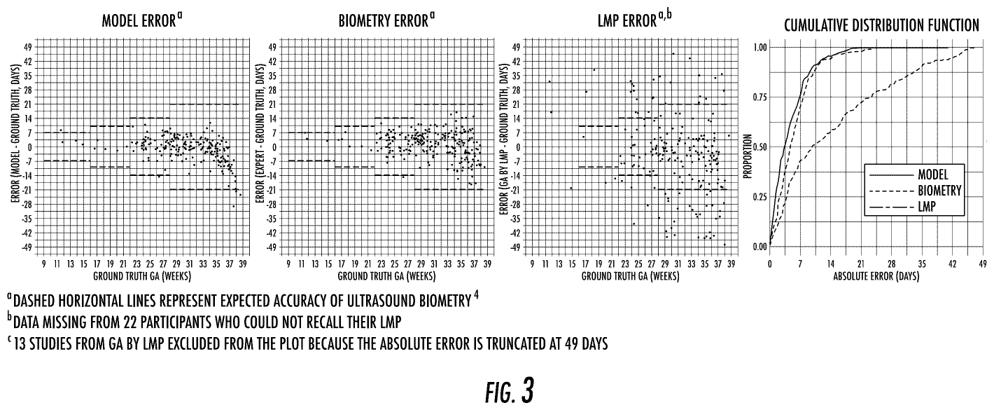

Table 3 and FIG. 3 illustrate results of gestational age predictions made by the trained machine learning model as compared to predictions made by un-trained users (i.e., “novices”) for a novice test set. The novice test set contains 249 sets of blind sweeps obtained on a low-cost, battery-powered device by an untrained user. As above, we compared model estimates to biometry obtained by a trained sonographer on commercial ultrasound. But we also compared the model estimates to the gestational age that would have been calculated had only the LMP been available (as is overwhelmingly the case in Zambia). In the novice test set, the model and biometry performed similarly: overall model MAE 4.9 days (SE 0.29) versus biometry MAE 5.4 days (SE 0.28); difference-0.6 days (95% CI: 1.3, 0.1); p=0.11). However, when compared to LMP, the model was clearly superior: model MAE 4.9 days (SE 0.29) versus LMP MAE 17.4 days (SE 1.17); difference −12.7 days (95% CI: −15.0, −10.3); p<0.001). Based on the empirical CDF, the proportion of study scans that were correctly classified within 7 days was substantially higher for the model than for LMP (71.9% vs 40.1%; difference 36.1% [95% CI 28.0%, 44.2%]; p<0.001). The model similarly outperformed LMP using a 14-day classification window (94.8% vs 55.1%; difference 40.5% [95% CI 33.9%, 47.1%]; p<0.001).

TABLE 3
Gestational age estimation of deep learning model
compared to trained sonographer in the novice test set
Novice Test Set (n = 249) ^{a}

| | | | | Difference | Difference |
|---|---|---|---|---|---|
| | | | | Model vs | Model vs. |
| | | | | Biometry | LMP^{b} |
| | Model | Biometry | LMP^{b} | (95% CI) | (95% CI) |
| Mean | 4.9 | 5.4 | 17.4 | −0.6 | −12.7 |
| Absolute | (0.29) | (0.28) | (1.17) | (−1.3, 0.1) | (−15.0, −10.3) |
| Error (SE), | | | | | |
| days | | | | | |
| Root Mean | 6.7 | 7.0 | 24.7 | −0.3 | −18.1 |
| Square | (0.44) | (0.54) | (1.68) | (−1.7, 1.0) | (−21.5, −14.7) |
| Error, | | | | | |
| days | | | | | |
| 1^{st} and 2^{nd} | 3.7 | 4.2 | 17.2 | −0.4 | −13.7 |
| trimester^{cd} | (0.39) | (0.38) | (2.25) | (−1.2, 0.3) | (−18.4, −9.1) |
| MAE (SE), | | | | | |
| days | | | | | |
| 3^{rd} | 5.3 | 5.9 | 17.5 | −0.6 | −12.3 |
| trimester^{c} | (0.37) | (0.36) | (1.37) | (−1.6, 0.3) | (−15.1, −9.5) |
| MAE(SE), | | | | | |
| days | | | | | |
| Percent | 75.1 | 71.9 | 40.1 | 3.2 | 36.1 |
| Absolute | (2.7) | (2.8) | (3.3) | (−3.3, 9.7) | (28.0, 44.2) |
| Error <7 | | | | | |
| days (SE) | | | | | |
| Percent | 95.6 | 94.8 | 55.1 | 0.8 | 40.5 |
| Absolute | (1.3) | (1.4) | (3.3) | (−2.9, 4.5) | (33.9, 47.1) |
| Error <14 | | | | | |
| days (SE) | | | | | |

^{a} The novice test set comprises all participants who contributed at least one set of blind sweeps performed by a novice user on a low-cost, battery-powered device; all participants enrolled in Zambia; expert biometry was performed by a sonographer on a commercial machine.
^{b}22 participants who could not recall their last menstrual period are excluded.
^{c}Trimesters defined as ≤97 days, 98-195 days, ≥196 days.
^{d}Only 2 studies in the 1^{st} trimester; 69 studies in the 2^{nd} trimester. MAE = mean absolute error; SE = standard error; CI = confidence interval; LMP = last menstrual period

### DISCUSSION

Quality Obstetric Care Requires Accurate Knowledge of Gestational Age. We built a deep learning model that can perform this critical assessment from blindly obtained ultrasound sweeps of the gravid abdomen. Expressed as mean absolute error or as the proportion of estimates that falls within 7 or 14 days of a previously defined ground truth gestational age, the model performance is superior to that of a trained sonographer performing fetal biometry on the same day. Results were consistent across geographical sites and supported in a test set of women who conceived by IVF (whose gestational age is unequivocal) and in a test set of women from whom the ultrasound blind sweeps were obtained by a novice provider using a low-cost, battery powered device.

This research addresses a critical shortcoming in the delivery of obstetrical care in low- and middle-income countries. The Lusaka public sector is typical of care systems across the African sub-continent and parts of Asia in that few women have access to ultrasound pregnancy dating and the median gestational age at presentation is 23 weeks (IQR 19, 26). This means that each year in the city of Lusaka, more than 100,000 pregnancies must be managed with an unacceptably low level of gestational age precision (FIG. 3). The availability of a resource-appropriate technology that could accurately assign gestational age in the late second and third trimesters could be transformative in Lusaka and similar settings around the world.

Recent years have seen an explosion in the application of deep learning to healthcare, particularly the interpretation of medical images. Successful projects to date have leveraged extant clinical datasets and trained machine learning algorithms on ideally-obtained single images (e.g., ocular fundus, breast, skin) that have been annotated by human experts. In contrast, our study collects thousands of images from each participant in the form of blind sweeps. Each cineloop frame in the sweep is itself a two-dimensional ultrasound image that is provided to the neural network during training. Although most of these frames would be considered clinically sub-optimal views, the sheer number of them (more than 21 million) provides a comprehensive picture of the developing fetus from every conceivable angle throughout gestation. Considered as individual images rather than participants or studies or sweeps, our training set is two orders of magnitude larger than most of the prior high-profile applications of deep learning to medical imaging. This may explain why the model so consistently outperforms expert biometry even when some of our training data include studies from women who present late for care and whose clinically established gestational age may be subject to measurement error.

Strengths of this study include its prospective nature, bespoke blind sweep sonography procedures, and inclusion of women from both high- and low-resource health care systems. We used several different makes and models of ultrasound scanners for data collection, a feature that likely bolsters the model's generalizability. Although we did not deliberately impose a lower gestational age limit on enrollment, our dataset includes very few scans at <9 gestational weeks and we thus are unable to make estimates below this threshold. Data were similarly sparse beyond 37 weeks (term gestation) and the model appears to systematically underestimate gestational age beyond this point in the novice test set. We note however that this limitation seems likely to affect only a minority of women-those who seek prenatal care but who have no visits between 9 and 37 weeks. From our prior population-based study of 115,552 pregnancies in Lusaka, under 1% of women would meet these criteria. Finally, we acknowledge that our blind sweep approach would be a sea change and might be seen as a threat to obstetric ultrasound capacity building in low-resource settings. Nonetheless, our data suggest that at least for gestational age estimation, capacity building efforts may be better directed elsewhere, as our approach is robust, and because it would be made freely available, affordable and scalable.

Beyond gestational age determination, obstetrical ultrasound can diagnose a wide range of conditions that may result in preventable morbidity or death, including ectopic pregnancy, multiple gestation, placenta previa, fetal demise, growth restriction, disorders of amniotic fluid volume, abnormal fetal blood flow, malpresentation, and fetal anatomic anomalies. Whether any of these diagnoses would be amenable to a deep learning approach is unknown but ongoing vibrant research at the intersection of artificial intelligence and obstetric sonography is promising. Given the ever-expanding computational capacity of mobile devices and the real advances that have been made in low-cost sonography, it seems only a matter of time until the world's most remote and under-resourced obstetrical services have access to the full diagnostic power of ultrasound.

### Ethical Approvals

This study protocol was approved by the University of North Carolina Institutional Review Board, the University of Zambia Biomedical Research Ethics Committee, and the Zambia National Health Research Authority prior to initiation.

### Appendix

### Section 1: Technical Methods for the Deep Learning Model

### Model Architecture

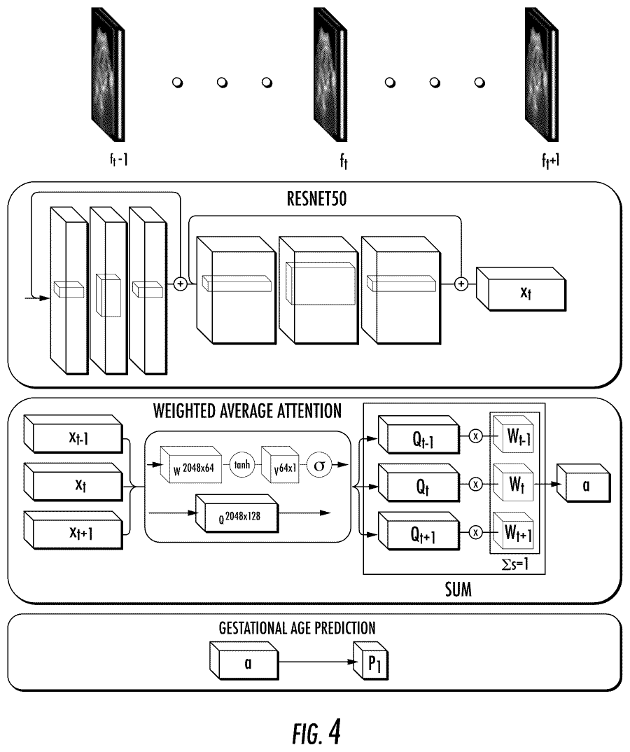

One example model architecture is graphically illustrated in FIG. 4 and includes the following two modules: Feature Extraction Module and Weighted Average Attention Module.

### Feature Extraction Module

During training, each frame is processed using ResNet-50 architecture initialized with weights trained on the ImageNet data set, this step yields a feature vector of size 2048 for each frame. The extracted features are then analyzed via our Weighted Average Attention (WAA) Module described in the following section. While a pre-trained network is used for feature extraction, the weights in ResNet-50 are fine-tuned together with the other parameters in the model during training.

### Weighted Average Attention Module

The functional form of our attention module is motivated by the additive Bahdanau attention. Our Weighted Average Attention (WAA) Module has 3 trainable parameters V, W, and Q as defined here:

$${w}_{t} = f ( {x}_{t} ) = σ ( {V}^{T} tanh ( W ( {x}_{t} ) ) ) \qquad ( 1 )$$

$${s}_{t} = \frac{{w}_{t}}{{\sum }_{u}^{N} {w}_{u}} \qquad ( 2 )$$

$$a = {\sum }_{t}^{N} {s}_{t} \cdot Q ( {x}_{t} ) \qquad ( 3 )$$

where xₜ is the output features from the feature extraction module. The attention module comprises W, which is a linear dense layer that outputs a vector of size 64, followed by the hyperbolic tangent activation function and finally a dot product with V to map to a single scalar value Wt between zero and one for each frame, where the scalar value for wt is determined by the sigmoid function denoted by σ in Equation 1. Equation 2 computes a weighted score sₜ on the time dimension so that Σs=1.

The parameters of the weighted average attention module are jointly trained with the other parts of the model. The attention mechanism described above allows the model to focus on frames of the input sequence that contain fetal structures and maximize the gestational age prediction power. The output of this attention module is a weighted sum of the features from the input frames and computed as shown in Equation 3 where Q is another dense layer which reduces the dimension of the feature vector xₜ from 2048 to 128. The weighted sum computation allows arbitrary sequence length and enables our model to make predictions based on a single or multiple frames.

Finally, a single linear layer takes variable a in Equation 3 as input and outputs the gestational age estimate.

### Training Procedure

In a pre-processing step, the blind sweeps are re-sampled to a common space with spacing of 0.75 mm/pixel and image dimensions of 256×256 pixels (i.e., physical size of 192×192 mm). To ensure that our network is learning solely from ultrasound image features, we mask and crop text that could bias learning.

The number of individual frames comprising each blind sweep cineloop is left unchanged.

Each 10-second blind sweep cineloop can contain 600 or more frames. Because training with all available frames in longer sequences can be computationally intense, we randomly select 50 frames from each sweep. We also apply the following additional processing to each frame from the blind sweep: padding to 288×288 image, and then 256×256 random cropping. The images are then loaded into the range [0, 1] and standard normalization (mean subtraction and standard deviation division by channel; both from ImageNet) is applied as required for the input of ResNet-50.

We use the adaptive moment estimation (ADAM) optimization algorithm with a learning rate of 10⁻⁴ and a batch size of 24 blind sweeps. Each training epoch contains 5000 batches in which weighted sampling is used to sample blind sweeps into each batch to handle the imbalanced distribution of gestational age. The loss function is Mean Absolute Error (MAE). We use an early stopping procedure and track the MAE of the studies in the tuning set, where for a given study all frames are concatenated to form a single sequence and used for prediction. When the tuning set MAE does not improve over 10 epochs, we stop the training and save the best model as evaluated by the tuning set MAE.

### Inference

At inference, all available blind sweeps for a study are concatenated and all available frames are used for predictions. This differs from the training procedures, where only a random subset of frames from a single blind sweep is input to the model. No data augmentation is applied and the full concatenated sequence of 256×256 frames is provided to the model. In a manner similar to that described above for training, each frame is loaded into the range [0, 1] and normalization is applied.

### Section 2: Exemplary Model Architecture

FIG. 4 illustrates one example of an architecture for a trained machine learning model for predicting gestational age from blind sweep ultrasound data. In the example illustrated in FIG. 4, feature extraction from each frame of ultrasound sweep data is performed using a RESNET50 architecture. During training, the RESNET50 is initialized using weights pre-trained on the ImageNet dataset. Each feature vector xₜ with dimension 2048 is used as input to our Weighted Average Attention Layer where a score w [0-1] is assigned to a frame using two fully connected layers (W, V). The dimension of each feature vector is reduced to 128 using a fully connected layer (Q). We use the scores to compute a weighted sum vector a which summarizes any input sequence with variable number of frames. Finally, a fully connected layer (P) estimates the gestational age.

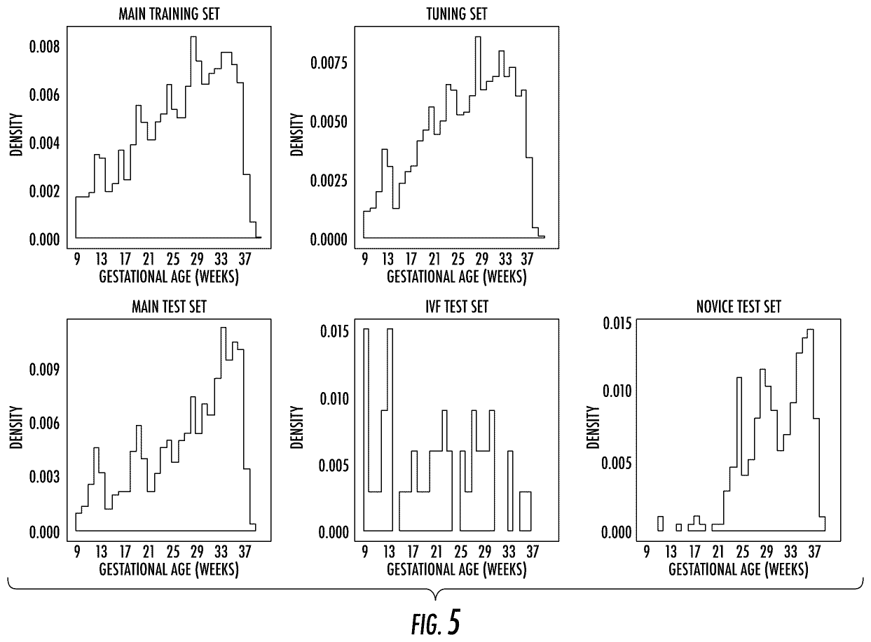

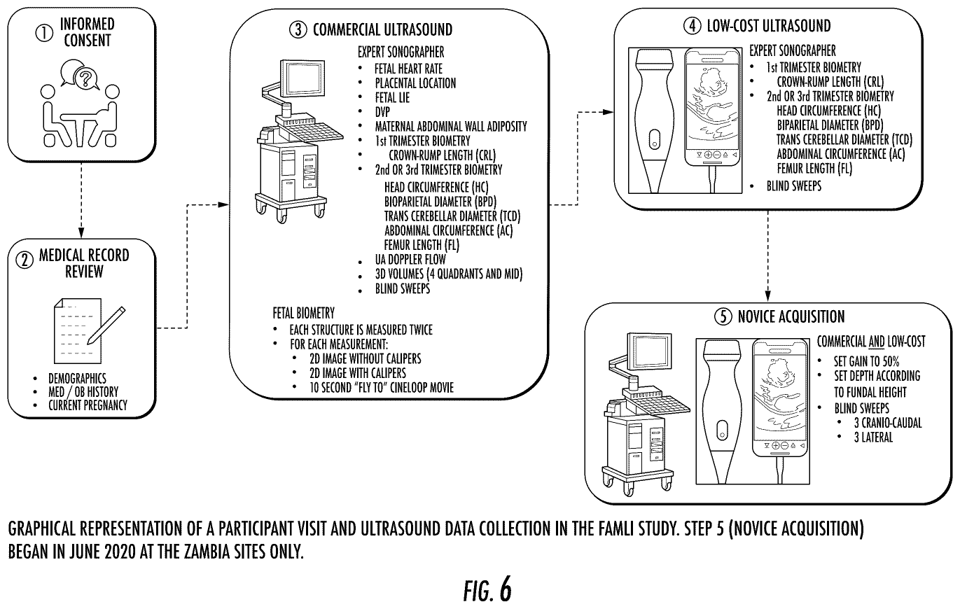

FIG. 5 illustrates the gestational age distribution of the training and testing sets. FIG. 6 is a graphical representation of a participant visit and ultrasound data collection in the FAMLI study. Step 5 (novice acquisition) began in June 2020 at the Zambia sites only.

### Section 3: Supplemental Tables

TABLE 4
Ultrasound Devices Used

| Ultrasound | Training | Tuning | Main | IVF | Novice |
|---|---|---|---|---|---|
| Make and | Set | Set | Test Set | Test Set | Test Set |
| Model | N = 4,770 | N = 1,188 | N = 716 | N = 47 | N = 249 |
| Butterfly iQ | 3833 | 941 | 10 | 10 | 249 |
| GE LOGIQ | 83 | 17 | 0 | 0 | 0 |
| C3 Premium | | | | | |
| GE LOGIQ e | 136 | 35 | 0 | 0 | 0 |
| Sonosite | 1955 | 457 | 219 | 0 | 0 |
| Mturbo | | | | | |
| GE Voluson E8 | 801 | 236 | 188 | 17 | 0 |
| GE Voluson S6 | 1712 | 417 | 299 | 30 | 10 |

Each participant study visit involves data collection with both a commercial and low-cost device. We limited the test sets to a single device (Main test set and IVF test set has commercial device only; Novice test set has low-cost device only). We did not impose this limitation on the training and tuning sets (i.e., during training a single participant study could contribute blind sweep cineloops from two devices.) Butterfly=Butterfly Network, Inc Guilford, CT, USA; GE=General Electric Healthcare, Zipf, Austria; Sonosite=Sonosite Inc, Bothell, WA, USA.

TABLE 5
Gestational age estimation of deep learning model compared
to expert sonographer - sensitivity analysis

| | Main Test Set (n = 1278)^{a} | | | IVF Test Set (n = 79)^{b} | | |
|---|---|---|---|---|---|---|
| | | | Difference | | | Difference |
| | Model | Biometry | (95% CI) | Model | Biometry | (95% CI) |
| Mean | 3.9 | 4.7 | −0.8 | 3.0 | 3.6 | −0.6 |
| Absolute | (0.09) | (0.11) | (−1.1, − 0.6) | (0.26) | (0.38) | (−1.3, 0.1) |
| Error (SE), | | | | | | |
| days | | | | | | |
| Root Mean | 5.1 | 6.2 | −1.1 | 3.8 | 4.9 | −1.1 |
| Square | (0.13) | (0.15) | (−1.4, − 0.8) | (0.32) | (0.47) | (−2.0, −0.2) |
| Error, | | | | | | |
| days | | | | | | |
| 1^{st} | 2.3 | 2.2 | 0.1 | 2.2 | 2.4 | — |
| trimester^{c} | (0.17) | (0.18) | (−0.4, 0.5) | (0.34) | (0.43) | |
| Mean Absolute | | | | | | |
| Error (SE), | | | | | | |
| days | | | | | | |
| 2^{nd} | 3.1 | 3.5 | −0.4 | 2.5 | 3.0 | — |
| trimester | (0.11) | (0.13) | (−0.7, −0.2) | (0.30) | (0.54) | |
| Mean Absolute | | | | | | |
| Error (SE), | | | | | | |
| days | | | | | | |
| 3^{rd} | 4.8 | 6.1 | −1.3 | 4.2 | 5.1 | — |
| trimester | (0.15) | (0.18) | (−1.7, −0.9) | (0.58) | (0.76) | |
| Mean Absolute | | | | | | |
| Error (SE), | | | | | | |
| days | | | | | | |
| Absolute | 85.4 | 77.3 | 8.1 | 92.4 | 84.8 | — |
| Error <7 | (1.0) | (1.2) | (5.6, 10.7) | (3.0) | (4.0) | |
| days (SE), | | | | | | |
| % | | | | | | |
| Absolute | 98.7 | 96.5 | 2.3 | 100.0 | 100.0 | — |
| Error <14 | (0.3) | (0.5) | (1.1, 3.4) | | | |
| days (SE), | | | | | | |
| % | | | | | | |
| North | 3.6 | 4.1 | −0.5 | — | — | — |
| Carolina | (0.12) | (0.14) | (−0.7, −0.2) | | | |
| Mean | | | | | | |
| Absolute | | | | | | |
| Error (SE), | | | | | | |
| days | | | | | | |
| Zambia | 4.2 | 5.5 | −1.3 | — | — | — |
| Mean | (0.15) | (0.18) | (−1.7, −0.9) | | | |
| Absolute | | | | | | |
| Error (SE), | | | | | | |
| days | | | | | | |

Our primary analyses limited test sets to a single ultrasound study per participant. This sensitivity analysis allows participants to contribute more than one study to their test set.
a The main test set comprises a 30% random sample of participants who are dated by a prior ultrasound and who are not included in the IVF or novice test sets; participants enrolled in either North Carolina or Zambia; blind sweeps and fetal biometry were collected by a sonographer on a commercial ultrasound machine.
b The IVF test set comprises all studies conducted in women who conceived by in vitro fertilization; all participants were enrolled in North Carolina; blind sweeps and fetal biometry were collected by a sonographer on a commercial ultrasound machine.
c Trimesters defined as ≤97 days, 98-195 days, ≥196 days.
   SE=standard error; CI=confidence interval; LMP=last menstrual period

TABLE 6
Gestational age estimation of deep learning model compared to expert sonographer - sensitivity analysis
Novice Test Set (n = 330)^{a}

| | | | | | | | Difference | | Difference | |
|---|---|---|---|---|---|---|---|---|---|---|
| | | | | | | | Model vs | | Model vs | |
| | | | | | | | Expert | | LMP^{b} | |
| | Model | | Biometry | | LMP^{b} | | (95% CI) | | (95% CI) | |
| Mean | 5.0 | (0.27) | 5.5 | (0.26) | 17.9 | (1.06) | −0.5 | (−1.1, 0.1) | −13.1 | (−15.2, −11.0) |
| Absolute | | | | | | | | | | |
| Error | | | | | | | | | | |
| (SE), | | | | | | | | | | |
| days | | | | | | | | | | |
| Root | 7.0 | (0.41) | 7.3 | (0.49) | 25.6 | (1.55) | −0.3 | (−1.3, 0.8) | −18.8 | (−21.9, −15.6) |
| Mean | | | | | | | | | | |
| Square | | | | | | | | | | |
| Error, | | | | | | | | | | |
| days | | | | | | | | | | |
| 1^{st} and 2^{nd} | 3.9 | (0.37) | 3.9 | (0.33) | 16.5 | (1.90) | −0.0 | (−0.7, 0.7) | −13.1 | (−17.0, −9.2) |
| trimester^{cd} | | | | | | | | | | |
| Mean | | | | | | | | | | |
| Absolute | | | | | | | | | | |
| Error | | | | | | | | | | |
| (SE), | | | | | | | | | | |
| days | | | | | | | | | | |
| 3^{rd} | 5.4 | (0.34) | 6.1 | (0.33) | 18.4 | (1.28) | −0.7 | (−1.5, 0.1) | −13.1 | (−15.6, −10.6) |
| trimester | | | | | | | | | | |
| Mean | | | | | | | | | | |
| Absolute | | | | | | | | | | |
| Error | | | | | | | | | | |
| (SE), | | | | | | | | | | |
| days | | | | | | | | | | |
| Absolute | 74.5 | (2.4) | 70.9 | (2.5) | 39.5 | (2.8) | 3.6 | (−2.2, 9.5) | 37.2 | (30.1, 44.2) |
| Error < 7 | | | | | | | | | | |
| days | | | | | | | | | | |
| (SE), % | | | | | | | | | | |
| Absolute | 93.9 | (1.3) | 94.2 | (1.3) | 54.7 | (2.9) | −0.3 | (−3.7, 3.1) | 39.9 | (34.0, 45.7) |
| Error < 14 | | | | | | | | | | |
| days | | | | | | | | | | |
| (SE), % | | | | | | | | | | |

Our primary analyses limited test sets to a single ultrasound study per participant. This sensitivity analysis allows participants to contribute more than one study to their test set.
a The novice test set comprises all participants who contributed at least one set of blind sweeps performed by a novice user on a low-cost, battery powered device; all participants enrolled in Zambia; expert biometry was performed by a sonographer on a commercial machine.
b 22 participants who could not recall their last menstrual period are excluded.
c Trimesters defined as ≤97 days, 98-195 days, ≥196 days.
d Only 2 studies in the 1^{st} trimester; 88 studies in the 2^{nd} trimester.
   SE=standard error; CI=confidence interval; LMP=last menstrual period

### Section 4: Use of Trained Machine Learning Model to Estimate Gestational Age

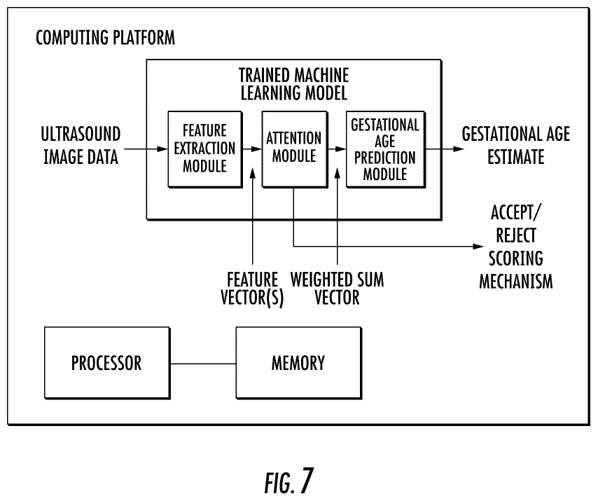

The model illustrated in FIG. 4 is trained using ultrasound image data and the ground truth gestational age data described above until the error function used to evaluate accuracy of the gestational age estimates stops decreasing. The result is a trained machine learning model with the same modules illustrated in FIG. 4 that can be used to estimate gestational age from ultrasound image data of a human fetus with an unknown gestational age. FIG. 7 illustrates the use of a trained machine learning model to estimate gestational age. In FIG. 7, the trained machine learning model includes the feature extraction module, the attention module, and the gestational age prediction module described above in FIG. 4, where each module has been trained using an ultrasound image dataset with known gestational ages as the ground truth data. The trained machine learning model may be implemented on a computing platform including at least one processor and a memory. The trained machine learning model may, in one example, be implemented using computer executable instructions stored in the memory which cause the processor to perform steps that implement the trained machine learning module. The computing platform may, in one example, be a mobile device or a cloud computing construct that receives ultrasound image data from a user via an application executing on a mobile device, propagates the ultrasound image data through the trained machine learning model to produce the gestational age estimate and provides the gestational age estimate to the application executing on the mobile device for display to the user.

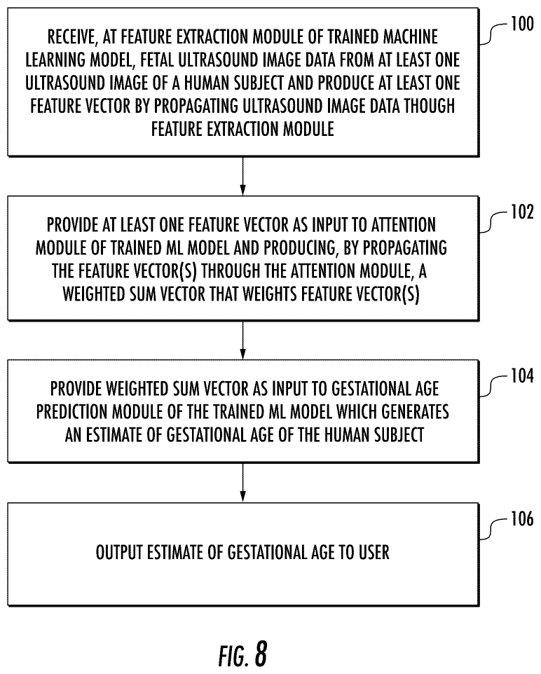

FIG. 8 illustrates a process for using the trained machine learning model in FIG. 7 to obtain an estimate of gestational age. Referring to FIG. 8, in step 100, the process includes receiving, at a feature extraction module of a trained machine learning model, fetal ultrasound image data from at least one image of a human subject (i.e., a human fetus), and producing, by propagating the ultrasound image data through the feature extraction module, at least one feature vector from the ultrasound image data. For example, to initiate the process for gestational age estimation, a user, such as a nurse or midwife, obtains ultrasound image data of a gravid abdomen in which the human subject has an unknown gestational age. In one example, the ultrasound image data may include the ultrasound sweep data described above. However, the subject matter described herein is not limited to using ultrasound sweep data. In an alternate example ultrasound image data from one or more still ultrasound images may be used without requiring video from continuous sweeps of the ultrasound probe.

If the ultrasound image data is implemented in a cloud or mobile computing environment, the ultrasound image data may be uploaded to the cloud computing environment via an application on a mobile device, such as a mobile phone.

The result of propagating each frame of ultrasound image data through the feature extraction module is a feature vector for each frame of ultrasound image data. The feature extraction module comprises a convolutional neural network that produces a feature vector for each frame. Elements of the feature vector encode image characteristics that are useful in determining fetal gestational age.

In step 102, the process includes providing the feature vector for each frame of image data as input to an attention module of the trained machine learning model and producing, by propagating the feature vector(s) through the attention module, a weighted sum vector. In general, the purpose of the attention module is to promote features that are good predictors of gestational age and minimize noise. In one example, the attention module is a weighted average attention module that reduces the dimensionality of each feature vector, weights the features in each vector according to their relative importance in estimating gestational age, generates a single unique value or score per frame (attention score), and generates a sum based on the scores and the reduced dimensionality feature vectors.

For a given sequence of ultrasound image frames, the corresponding attention scores generated by the attention module can be used in a feedback/rejection mechanism to either prompt the user to repeat data collection or indicate that the model cannot produce a reliable gestational age estimate.

In step 104, the process includes providing the weighted sum vector as input to a gestational age prediction module of the trained machine learning mode, which generates, from the weighted sum vector, an estimate of gestational age of the subject. In one example, the gestational age prediction module comprises a linear prediction module weighted from the training, that takes the weighted sum vector as input, applies the weight(s), and produces an output, which In one example, indicates a gestational age in days. In one example, the gestational age prediction module produces a linear combination of the elements of the feature vector using learned weights to combine the elements.

In step 106, the process includes outputting the estimate of gestational age to a user. For example, if the trained machine learning model is implemented on a tablet computer, the model may be part of an application that outputs the estimate of gestational age to the user. In another example, if the trained machine learning model is implemented in a cloud network, the model may be part of a server that transmits the estimate of gestational age to a computing device, such as a mobile device, local to the user, and the computing device may display the estimate of gestational age to the user.

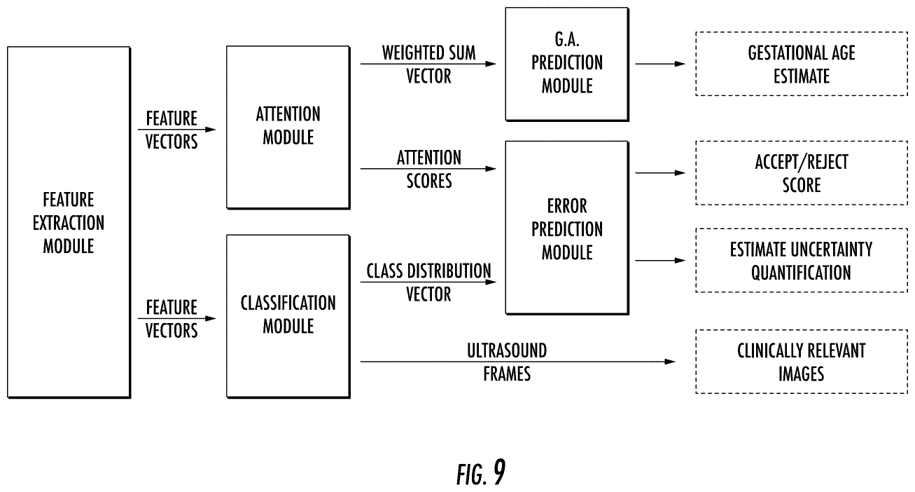

FIG. 9 is a block diagram illustrating an alternate trained machine learning model for estimating gestational age. The feature extraction module performs the same function described above with regard to FIGS. 4 and 7, i.e., producing a feature vector for each frame of ultrasound image data to be used for gestational age estimation. It also produces a separate feature vector for each frame that can be used for image classification. The attention module also performs the same function described above with regard to FIGS. 4 and 7 of producing a weighted sum vector and an attention score for each feature vector. The GA prediction module likewise performs the functions described above of outputting a gestational age estimate.

The classification module illustrated in FIG. 9 receives feature vectors output from feature extraction module. The classification module assigns each image in a series of ultrasound frames to one of many predefined classes (see Table 7). Some of these classes have been labeled by experts to correspond to clinically relevant images (e.g., a transthalamic view of the fetal head). The classification module may be used to output clinically relevant frames to a user. The classification module also creates a class distribution vector that describes the pattern or “fingerprint” of class membership for a given series of ultrasound frames.

The error prediction module receives as input a class distribution vector for a series of image frames and an attention score for each image frame in that series. It outputs an accept/reject score that can be used to prompt a user to collect additional data and/or to prevent an unreliable estimate from being output to the user. The error prediction module also outputs, in the form of a prediction interval, an estimate of uncertainty in the GA prediction for the series.

### Classification Module

Image Labeling Via Contrastive Learning and kMeans

Our unsupervised clustering approach is inspired by SimCLR¹. We generate image embeddings of dimension 128 and use kMeans² to automatically find image clusters.

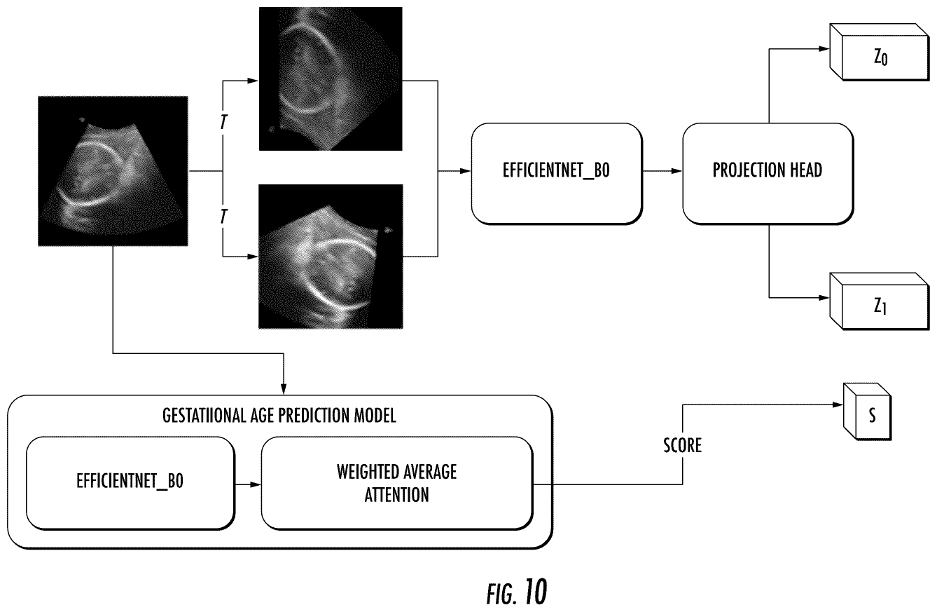

FIG. 10 shows the steps to create two embeddings of the same image under different transformations. We use the trained gestational age model (FIG. 4) to compute the image attention score. This scalar between [0, 1] is correlated to image quality for the computation of gestational age, however, the image content is unknown. In FIG. 10, a transformation T is applied twice to an input US frame (in this example, a fetal head). The transformed frames are projected to 128-dimension embeddings (Z₀, Z₁). The trained Attention module from the Gestational Age Prediction Model (FIG. 4) is used to produce an attention score for each frame. The embeddings and scores are used to compute the contrastive loss.

$$cos\_sim = \frac{A \cdot B}{‖ A ‖ ‖ B ‖} \qquad ( 1 )$$

$$S = {sort}_{usc} \qquad ( 2 )$$

$$W = w * {( 1 - x )}^{2} , x ∈ [ 0 , 1 ] , w = 16 \qquad ( 3 )$$

$$similarity = {\sum }_{t}^{N} {( 1 - cos\_sim ( {𝓏}_{0} , {𝓏}_{1} ) )}^{2} \qquad ( 4 )$$

$$contrastive = {\sum }_{t}^{N} {( W * S ( cos\_sim ( {𝓏}_{0 r} , {𝓏}_{1} ) ) )}^{2} \qquad ( 5 )$$

$$north = {\sum }_{t}^{N} {( s - ( 1 - cos\_sim ( {𝓏}_{0} , N ) ) + s - ( 1 - cos\_sim ( {𝓏}_{1} , N ) ) )}^{2} \qquad ( 6 )$$

$$L = similarity + contrastive + north \qquad ( 7 )$$

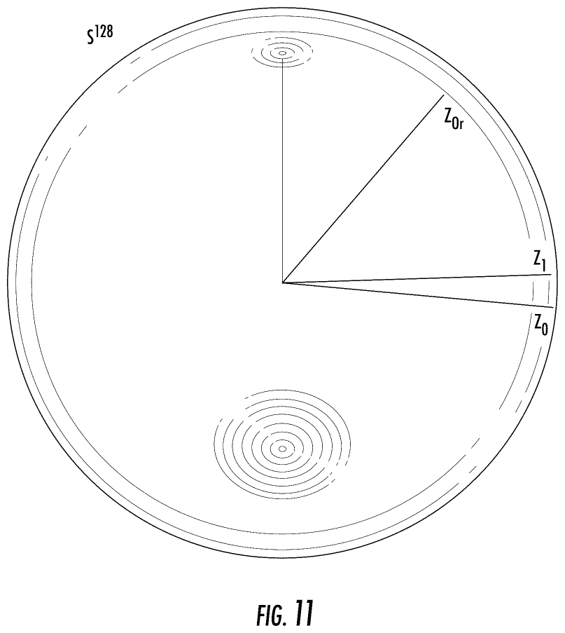

FIG. 11 is a diagram illustrating a 128-dimension unit hypersphere. The embeddings Z₀ and Z₁ correspond to the same image under different transformations. While Z₀ᵣ corresponds to a different image in the same batch. The loss function is composed of 3 terms: similarity, contrastive, and north. The similarity term uses the cosine similarity and aims to minimize the angle between embeddings of the same image under 2 different transformations. The cosine similarity is equal to 1 if the vectors are colinear, 0 if the vectors are orthogonal, and −1 if they point in opposite directions. The image transformations include random intensity (color jitter) and random rotation or random resize crop.

The contrastive term starts by randomizing z₀→z₀ᵣ images in the batch (the batch size during training is 256) and computes the cosine similarity against z₁. The resulting values are then sorted in ascending order, i.e., the most orthogonal/different will be first. Finally, we scale the resulting values using W(1−x)² where W is a hyperparameter set to 16. The loss function encourages different images to be far apart in the embedding space while similar images to be closer.

Finally, the north term uses the score scalar s given by the GA model to further organize the embedding space. It pushes images that have important fetal anatomy to be closer to the equator of the hypersphere while images without meaningful information to be closer to the north pole, i.e., the vector of zeros with 1 in the final dimension.

The data split is the same as the splits used for training the gestational age prediction model.

The contrastive model is trained for 177 epochs, we use the AdamW optimizer with learning rate 1e⁻⁴ and use the early stopping criteria with patience 30. After the training is done, we use kMeans to automatically find images clusters. The image clusters are then analyzed by expert sonographers and labels are assigned to each cluster. The unsupervised clustering method identifies fetal structures of different quality notably head images with varying quality.

### Classification

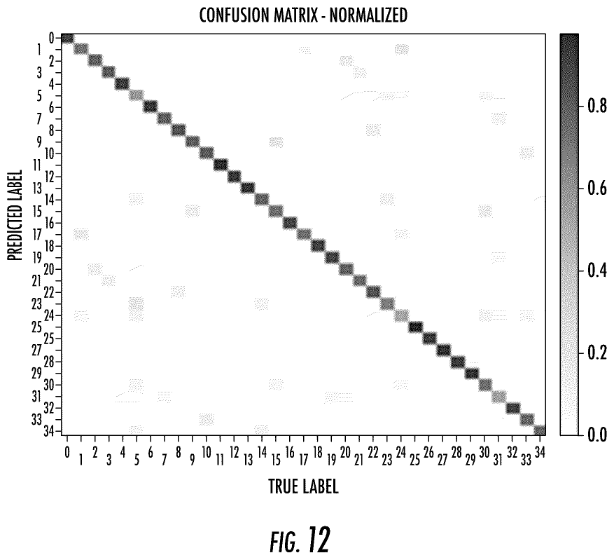

The labels produced by the unsupervised method described above are used to train a classifier. We use 35 classes and the cross-entropy loss for a multiclass classification problem. The data used for the classification task uses the test split only. The test split is further subdivided following a 0.7, 0.1, and 0.2 splits for training, validation, and testing. The fetal structures include head, abdomen, femur, placenta, gestational sac, low/high quality head images. FIG. 12 shows the normalized confusion matrix and Table 7 the classification report with an average f1-score of 0.8 for the classification task.

With this image classification model, we provide the possibility to automatically assign labels to the blind sweep videos used by the gestational age prediction model. This module provides the possibility to display clinically relevant frames to the experts, while hiding frames that are not meaningful for an expert.

TABLE 7
Classification report

| Class | precision | recall | f1-score | support |
|---|---|---|---|---|
| 0 | 0.82 | 0.9 | 0.86 | 45810 |
| 1 | 0.96 | 0.7 | 0.81 | 143397 |
| 2 | 0.76 | 0.77 | 0.77 | 51610 |
| 3 | 0.56 | 0.8 | 0.65 | 20486 |
| 4 | 0.83 | 0.9 | 0.86 | 43656 |
| 5 | 0.25 | 0.56 | 0.35 | 28477 |
| 6 | 0.96 | 0.92 | 0.94 | 77591 |
| 7 | 0.86 | 0.78 | 0.82 | 75263 |
| 8 | 0.73 | 0.83 | 0.78 | 45015 |
| 9 | 0.89 | 0.78 | 0.84 | 98890 |
| 10 | 0.78 | 0.79 | 0.79 | 57659 |
| 11 | 0.88 | 0.96 | 0.92 | 45390 |
| 12 | 0.87 | 0.92 | 0.89 | 58743 |
| 13 | 0.93 | 0.94 | 0.93 | 59071 |
| 14 | 0.92 | 0.77 | 0.83 | 139529 |
| 15 | 0.71 | 0.72 | 0.72 | 71078 |
| 16 | 0.91 | 0.87 | 0.89 | 39172 |
| 17 | 0.5 | 0.71 | 0.59 | 14327 |
| 18 | 0.91 | 0.89 | 0.9 | 51943 |
| 19 | 0.86 | 0.87 | 0.87 | 62580 |
| 20 | 0.78 | 0.77 | 0.78 | 71035 |
| 21 | 0.91 | 0.74 | 0.82 | 90914 |
| 22 | 0.85 | 0.83 | 0.84 | 80992 |
| 23 | 0.77 | 0.67 | 0.72 | 94372 |
| 24 | 0.31 | 0.51 | 0.38 | 33968 |
| 25 | 0.92 | 0.98 | 0.95 | 40400 |
| 26 | 0.92 | 0.91 | 0.91 | 38539 |
| 27 | 0.85 | 0.94 | 0.89 | 41017 |
| 28 | 0.86 | 0.91 | 0.88 | 54227 |
| 29 | 0.89 | 0.94 | 0.92 | 65910 |
| 30 | 0.32 | 0.72 | 0.44 | 15053 |
| 31 | 0.67 | 0.53 | 0.59 | 77498 |
| 32 | 0.93 | 0.89 | 0.91 | 51995 |
| 33 | 0.74 | 0.75 | 0.75 | 63148 |
| 34 | 0.24 | 0.76 | 0.37 | 3223 |
| accuracy | | | 0.8 | 2051978 |
| macro | 0.77 | 0.81 | 0.78 | 2051978 |
| avg | | | | |
| weighted | 0.83 | 0.8 | 0.81 | 2051978 |
| avg | | | | |

### Error Prediction Module

### Estimate Uncertainty Quantification

This module functions both as a quality control mechanism for the blind sweeps collected by the user and a user feedback mechanism. It takes as inputs the attention scores from the attention layer (GA model), the class distribution vector from the classification module, feature vectors from GA model and/or unsupervised clustering model, as well as the gestational age estimate (GA model). The module combines these inputs to assess the quality of information gathered from a series of ultrasound images.

This module performs two quality assessments tasks. First, it uses the attention scores and a thresholding operation to determine that a certain number of frames in the blind sweeps meet a minimum quality criterion to calculate the gestational age. Second, using the class distribution vector, it evaluates the quality (for gestational age prediction) of information collected in a series of ultrasound images. The module uses this quality assessment to construct an individualized prediction interval for the gestational age estimate produced by gestational age prediction module. The output of this module may include, for example, 1. a confidence score; 2. a prediction interval for the gestational age estimate; 3. an accept or reject decision by the module. Based on these outputs, the user may be prompted to repeat the blind sweeps collection procedure.

The disclosure of each of the following references is incorporated herein by reference in its entirety.

### REFERENCES

1. World Health Organization. WHO recommendations on antenatal care for a positive pregnancy experience (2016). Available at: https://www.who.int/reproductivehealth/publications/maternal_perinatal_health/anc-positivepregnancy-experience/en/Accessed 28 Jul. 2021.
2. Kramer M S, McLean F H, Boyd M E, Usher R H. The validity of gestational age estimation by menstrual dating in term, preterm, and post term gestations. Jama. 1988; 260(22): 3306-3308.
3. Matsumoto S, Nogami Y, Ohkuri S. Statistical studies on menstruation: a criticism on the definition of normal menstruation. Gunma J Med Sci 1962; 11:294-318.
4. American College of Obstetricians and Gynecologists, American Institute of Ultrasound in Medicine, Society for Maternal-Fetal Medicine. Committee Opinion No 700: Methods for Estimating the Due Date. Obstetrics and gynecology. 2017; 129(5): e150-e154.
5. Yadav H, Shah D, Sayed S, Horton S, Schroeder L F. Availability of essential diagnostics in ten low-income and middle-income countries: results from national health facility surveys. The Lancet Global health. 2021.
6. Marsh-Feiley G, Eadie L, Wilson P. Telesonography in emergency medicine: A systematic review. PloS one. 2018; 13(5): e0194840.
7. Becker D M, Tafoya C A, Becker S L, Kruger G H, Tafoya M J, Becker T K. The use of portable ultrasound devices in low- and middle-income countries: a systematic review of the literature. Trop Med Int Health. 2016; 21(3): 294-311.
8. Carin L, Pencina M J. On Deep Learning for Medical Image Analysis. In: Livingston E H, Lewis R J, eds. JAMA Guide to Statistics and Methods. New York, NY: McGraw-Hill Education; 2019.
9. Esteva A, Robicquet A, Ramsundar B, et al. A guide to deep learning in healthcare. Nature medicine. 2019; 25(1):24-29.
10. Chi B H, Vwalika B, Killam W P, et al. Implementation of the Zambia electronic perinatal record system for comprehensive prenatal and delivery care. International journal of gynecology and obstetrics: the official organ of the International Federation of Gynecology and Obstetrics. 2011; 113(2):131-136.
11. Price J T, Winston J, Vwalika B, et al. Quantifying bias between reported last menstrual period and ultrasonography estimates of gestational age in Lusaka, Zambia. International journal of gynecology and obstetrics: the official organ of the International Federation of Gynecology and obstetrics. 2019; 144(1):9-15.
12. Castillo M C, Fuseini N M, Rittenhouse K, et al. The Zambian Preterm Birth Prevention Study (ZAPPS): Cohort characteristics at enrollment. Gates Open Res. 2018; 2:25.
13. Price J T, Vwalika B, Rittenhouse K J, et al. Adverse birth outcomes and their clinical phenotypes in an urban Zambian cohort. Gates Open Res. 2019; 3:1533.
14. Zambian Ministry of Health. Annual Health Statistics Report 2017-2019; Available at https://www.moh.gov.zm/?wpfb_dl=159 (accessed 19 Oct. 2021). October 2020, Lusaka.
15. Vwalika B, Price J T, Rosenbaum A, Stringer J S A. Reducing the global burden of preterm births. The Lancet Global health. 2019; 7(4):e415.
16. Gulshan V, Peng L, Coram M, et al. Development and Validation of a Deep Learning Algorithm for Detection of Diabetic Retinopathy in Retinal Fundus Photographs. Jama. 2016; 316(22):2402-2410.
17. Milea D, Najjar R P, Zhubo J, et al. Artificial Intelligence to Detect Papilledema from Ocular Fundus Photographs. The New England journal of medicine. 2020; 382(18):1687-1695.
18. Mckinney S M, Sieniek M, Godbole V, et al. International evaluation of an AI system for breast cancer screening. Nature. 2020; 577(7788):89-94.
19. Esteva A, Kuprel B, Novoa R A, et al. Dermatologist level classification of skin cancer with deep neural networks. Nature. 2017; 542(7639):115-118.
20. Maraci M A, Yaqub M, Craik R, et al. Toward point-of-care ultrasound estimation of fetal gestational age from the trans-cerebellar diameter using CNN based ultrasound image analysis. J Med Imaging (Bellingham). 2020; 7(1):014501.
21. van den Heuvel T L A, Petros H, Santini S, de Korte C L, van Ginneken B. Automated Fetal Head Detection and Circumference Estimation from Free-Hand Ultrasound Sweeps Using Deep Learning in Resource-Limited Countries. Ultrasound Med Biol. 2019; 45(3):773-785.
22. He K, Zhang X, Ren S, Sun J. Deep Residual Learning for Image Recognition; 2016 IEEE Conference on Computer Vision and Pattern Recognition (CVPR), 2016, pp. 770-778, doi: 10.1109/CVPR.2016.90.
23. Deng J, Dong W, Socher R, Li L, Li K, Li F F. ImageNet: A large-scale hierarchical image database. 2009 IEEE Conference on Computer Vision and Pattern Recognition, 2009, pp. 248-255, doi: 10.1109/CVPR.2009.5206848.
24. Kanezaki A, Matsushita Y, Nishida Y. RotationNet for Joint Object Categorization and Unsupervised Pose Estimation from Multi-View Images. IEEE Trans Pattern Anal Mach Intell. 2021; 43(1): 269-283.
25. Su H, Maji S, Kalogerakis E, Learned-Miller E G. Multi-view Convolutional Neural Networks for 3D Shape Recognition. 2015 IEEE International Conference on Computer Vision (ICCV). 2015:945-953.
26. Ma C, Guo Y, Yang J, An W. Learning Multi-View Representation With LSTM for 3-D Shape Recognition and Retrieval. IEEE Transactions on Multimedia. 2019; 21:1169-1182.
27. Wang C, Pelillo M, Siddiqi K. Dominant Set Clustering and Pooling for Multi-View 3D Object Recognition. ArXiv. 2017; abs/1906.01592.
28. Bahdanau D, Chorowski J, Serdyuk D, Brakel P, Bengio Y. End-to-end attention-based large vocabulary speech recognition. 2016 IEEE International Conference on Acoustics, Speech and Signal Processing (ICASSP); 2016; Shanghai, China; DOI:10.1109/icassp.2016.7472618.
29. Kingma D P, Ba J. Adam: A Method for Stochastic Optimization. CoRR. 2015; abs/1412.6980; available at https://arxiv.org/abs/1412.6980v9 (accessed 23 Oct. 2021).
30. Yao Y, Rosasco L, Caponnetto A. On Early Stopping in Gradient Descent Learning. Constructive Approximation. 2007; 26(2):289-315.

It will be understood that various details of the subject matter described herein may be changed without departing from the scope of the subject matter described herein. Furthermore, the foregoing description is for the purpose of illustration only, and not for the purpose of limitation, as the subject matter described herein is defined by the claims as set forth hereinafter.

What is claimed is:

1. A method for estimating gestational age of a human fetus using a trained machine learning model with an attention function, the method comprising:
receiving, at a feature extraction module of a trained machine learning model, fetal ultrasound image data for at least one image of a human fetus, and producing, by propagating the ultrasound image data through the feature extraction module, at least one feature vector from the ultrasound image data; providing the at least one feature vector as input to an attention module of the trained machine learning model and producing, by propagating the feature vectors through the attention module, a weighted sum vector that aggregates and weights the feature vectors;
providing the weighted sum vector as input to a gestational age prediction module of the trained machine learning mode, which generates, from the weighted sum vector, an estimate of the gestational age of the human fetus; and
outputting the estimate of gestational age to a user.

2. The method of claim 1 wherein the ultrasound image data comprises video ultrasound image data obtained from sweeping an ultrasound probe across a gravid abdomen.

3. The method of claim 1 wherein the feature extraction module comprises convolutional neural network.

4. The method of claim 1 wherein the attention module comprises a weighted average attention module.

5. The method of claim 4 wherein the weighted average attention module weights the at least one feature vector based on relative importance of the features in the at least one feature vector in estimating gestational age.

6. The method of claim 5 wherein the weighted average attention module reduces dimensionality of the at least one feature vector.

7. The method of claim 1 wherein the attention module outputs an attention score for each attention vector indicative of a predictive quality of features in each feature vector.

8. The method of claim 7 comprising outputting the feature vectors from the feature extraction module to a classification module, and producing, using the classification module, a class distribution vector for each of the feature vectors.

9. The method of claim 8 comprising using the class distribution vector and the attention score for each feature vector to output a score indicative of a quality of the gestational age estimate.

10. The method of claim 8 comprising using the feature vector to generate an estimate of uncertainty for the gestational age estimate.

11. The method of claim 8 comprising selecting, using the class distribution vectors, clinically relevant images from the image frames.

12. A system for implementing the method of any one of claims 1-11.

13. A system for estimating gestational age of a human fetus using a trained machine learning model with an attention function, the system comprising:
at least one processor;
a trained machine learning module implemented using the at least one processor, the trained machine learning module including a feature extraction module, an attention module, and a gestational age prediction module;
the feature extraction module for receiving fetal ultrasound image data for at least one image of a human fetus, and producing, by propagating the ultrasound image data through the feature extraction module, at least one feature vector from the ultrasound image data, providing the at least one feature vector as input to the attention module;
the attention module for producing, by propagating the feature vectors through the attention module, a weighted sum vector that aggregates and weights the feature vectors and providing the weighted sum vector as input to the gestational age prediction module; and
the gestational age prediction module for generating, from the weighted sum vector, an estimate of the gestational age of the human fetus and outputting the estimate of gestational age to a user.

14. The system of claim 13 wherein the ultrasound image data comprises video ultrasound image data obtained from sweeping an ultrasound probe across a gravid abdomen.

15. The system of claim 13 wherein the feature extraction module comprises a convolutional neural network.

16. The system of claim 13 wherein the attention module comprises a weighted average attention module.

17. The system of claim 16 wherein the weighted average attention module weights the at least one feature vector based on relative importance of the features in the at least one feature vector in estimating gestational age.

18. The system of claim 17 wherein the weighted average attention module reduces dimensionality of the at least one feature vector.

19. The system of claim 13 wherein the attention module outputs an attention score for each attention vector indicative of a predictive quality of features in each feature vector.

20. The system of claim 19 wherein the trained machine learning model includes a classification module for receiving the feature vectors output from the feature extraction module and producing a class distribution vector for each of the feature vectors.

21. The system of claim 20 wherein the trained machine learning model includes an error prediction module for using the class distribution vector and the attention score for each feature vector to output a score indicative of a quality of the gestational age estimate.

22. The system of claim 20 wherein the trained machine learning model includes an error prediction module for using the feature vector to generate an estimate of uncertainty for the gestational age estimate.

23. The system of claim 20 wherein the classification module is configured to select, using the class distribution vectors, clinically relevant images from the image frames.

24. One or more non-transitory computer readable media comprising computer executable instructions that when executed by at least one processor of at least one computer control the at least one computer to implement the method of any of claims 1-11.
